(19) United States
(12) Patent Application Publication
(10) Pub. No.: US 2024/0325725 A1
(43) Pub. Date: Oct. 3, 2024

(54) GUIDEWIRE INSERTION AIDES FOR PERCUTANEOUS CIRCULATORY SUPPORT DEVICES

(71) Applicant: Boston Scientific Scimed, Inc., Maple Grove, MN (US)

(72) Inventors: Brice Lee Shireman, Maple Grove, MN (US); Jeffrey Matthew Lucas, Eden Prairie, MN (US); Brian R. Reynolds, Ramsey, MN (US); Qian Liu, Plymouth, MN (US)

(73) Assignee: Boston Scientific Scimed, Inc., Maple Grove, MN (US)

(21) Appl. No.: 18/617,010

(22) Filed: Mar. 26, 2024

Related U.S. Application Data
(60) Provisional application No. 63/455,931, filed on Mar. 30, 2023.

(51) Int. Cl. A61M 60/865; A61M 60/126; A61M 60/178; A61M 60/216; A61M 60/419; A61M 60/81

(52) U.S. Cl. CPC A61M 60/865; A61M 60/126; A61M 60/178; A61M 60/216; A61M 60/419; A61M 60/81; A61M 2205/04; A61M 2205/10; A61M 2209/00; A61M 2210/125

(57) ABSTRACT

A percutaneous circulatory support system includes a percutaneous circulatory support device having an impeller and a distal portion. A guidewire insertion aide is removably carried in the distal portion of the percutaneous circulatory support device. The guidewire insertion aide includes a tapered distal end portion initially in contact with the distal portion of the percutaneous circulatory support device. The guidewire insertion aide is configured to couple to a guidewire.

### CROSS-REFERENCE TO RELATED APPLICATIONS

This application claims the benefit of U.S. Provisional Patent Application Ser. No. 63/455,931, filed Mar. 30, 2023, which is incorporated herein by reference.

### TECHNICAL FIELD

The present disclosure relates to percutaneous circulatory support devices and associated components. More specifically, the present disclosure relates to guidewire insertion aides for percutaneous circulatory support devices.

### BACKGROUND

Percutaneous circulatory support devices such as blood pumps can provide transient support for up to approximately several weeks in patients with compromised heart function or cardiac output. Such devices are typically delivered to a patient's heart using a guidewire. More specifically, a distal end of the guidewire is inserted into the patient and positioned in the patient's heart while a proximal end of the guidewire remains outside of the patient. The proximal end of the guidewire is then inserted through a percutaneous circulatory support device such that the device is translatable along the guidewire to the patient's heart. However, inserting the guidewire through the percutaneous circulatory support device can damage delicate components of the device.

### SUMMARY

In an Example 1, a percutaneous circulatory support system includes a percutaneous circulatory support device having an impeller and a distal portion. The system further includes a guidewire insertion aide removably carried in the distal portion of the percutaneous circulatory support device. The guidewire insertion aide includes a tapered distal end portion initially in contact with the distal portion of the percutaneous circulatory support device. The guidewire insertion aide is configured to couple to a guidewire.

In an Example 2, the percutaneous circulatory support system of Example 1, wherein the distal portion of the percutaneous circulatory support device includes a cannula having a cannula lumen and a flexible distal extension including an extension lumen coupled to the cannula lumen. The tapered distal end portion of the guidewire insertion aide is initially disposed in the cannula lumen and in contact with the cannula.

In an Example 3, the percutaneous circulatory support system of Example 2, wherein the cannula includes a blood inlet and the percutaneous circulatory support device further includes a blood outlet. The guidewire insertion aide is configured to be removed from the percutaneous circulatory support device through the blood outlet.

In an Example 4, the percutaneous circulatory support system of any of Examples 1-2, wherein the percutaneous circulatory support device includes a blood inlet and a blood outlet, and the guidewire insertion aide is configured to be removed from the percutaneous circulatory support device through the blood outlet.

In an Example 5, the percutaneous circulatory support system of Example 4, wherein a proximal end portion of the guidewire insertion aide is disposed outwardly from the blood outlet.

In an Example 6, the percutaneous circulatory support system of any of Examples 1-5, wherein the guidewire insertion aide includes a through lumen.

In an Example 7, the percutaneous circulatory support system of any of Examples 1-5, wherein the guidewire insertion aide includes a blind lumen.

In an Example 8, the percutaneous circulatory support system of any of Examples 1-7, wherein the percutaneous circulatory support device further includes a housing, the impeller being carried in the housing, and the distal portion being coupled to the housing.

In an Example 9, a percutaneous circulatory support system includes a percutaneous circulatory support device having an impeller, a cannula including a distal adapter having a cannula lumen, and a distal extension coupled to the distal adapter. A guidewire insertion aide is removably carried in the cannula lumen. The guidewire insertion aide includes an enlarged coupling portion initially in contact with the distal adapter. The guidewire insertion aide is configured to couple to a guidewire.

In an Example 10, the percutaneous circulatory support system of Example 9, wherein the enlarged coupling portion is a distal end portion of the guidewire insertion aide.

In an Example 11, the percutaneous circulatory support system of any of Examples 9-10, wherein the percutaneous circulatory support device includes a blood inlet and a blood outlet, and the guidewire insertion aide is configured to be removed from the percutaneous circulatory support device through the blood outlet.

In an Example 12, the percutaneous circulatory support system of Example 11, wherein a proximal end portion of the guidewire insertion aide is disposed outwardly from the blood outlet.

In an Example 13, the percutaneous circulatory support system of any of Examples 9-12, wherein the guidewire insertion aide includes a through lumen.

In an Example 14, the percutaneous circulatory support system of any of Examples 9-12, wherein the guidewire insertion aide includes a blind lumen.

In an Example 15, the percutaneous circulatory support system of any of Examples 9-14, wherein the percutaneous circulatory support device further includes a housing, the impeller being carried in the housing, and the cannula being coupled to the housing.

In an Example 16, a percutaneous circulatory support system includes a percutaneous circulatory support device having a housing, an impeller carried in the housing, and a distal portion coupled to the housing. A guidewire insertion aide is removably carried in the housing and the distal portion of the percutaneous circulatory support device. The guidewire insertion aide includes a tapered distal end portion initially in contact with the distal portion of the percutaneous circulatory support device, and the guidewire insertion aide is configured to couple to a guidewire.

In an Example 17, the percutaneous circulatory support system of Example 16, wherein the distal portion of the percutaneous circulatory support device includes a cannula including a cannula lumen and a flexible distal extension including an extension lumen coupled to the cannula lumen. The tapered distal end portion of the guidewire insertion aide is initially disposed in the cannula lumen and in contact with the cannula.

In an Example 18, the percutaneous circulatory support system of Example 17, wherein the cannula includes a blood inlet and the housing includes a blood outlet, and the guidewire insertion aide is configured to be removed from the housing through the blood outlet.

In an Example 19, the percutaneous circulatory support system of Example 16, wherein the percutaneous circulatory support device includes a blood inlet and a blood outlet, and the guidewire insertion aide is configured to be removed from the housing through the blood outlet.

In an Example 20, the percutaneous circulatory support system of Example 19, wherein a proximal end portion of the guidewire insertion aide is disposed outwardly from the blood outlet.

In an Example 21, the percutaneous circulatory support system of Example 16, wherein the guidewire insertion aide includes a through lumen.

In an Example 22, the percutaneous circulatory support system of Example 16, wherein the guidewire insertion aide includes a blind lumen.

In an Example 23, a percutaneous circulatory support system includes a percutaneous circulatory support device having a housing, an impeller carried in the housing, a cannula coupled to the housing, the cannula having a distal adapter having a cannula lumen, and a distal extension coupled to the distal adapter. The system further includes a guidewire insertion aide removably carried in the housing and the cannula lumen. The guidewire insertion aide includes an enlarged coupling portion initially in contact with the distal adapter, and the guidewire insertion aide is configured to couple to a guidewire.

In an Example 24, the percutaneous circulatory support system of Example 23, wherein the enlarged coupling portion is a distal end portion of the guidewire insertion aide.

In an Example 25, the percutaneous circulatory support system of Example 23, wherein the percutaneous circulatory support device includes a blood inlet and a blood outlet, and the guidewire insertion aide is configured to be removed from the housing through the blood outlet.

In an Example 26, the percutaneous circulatory support system of Example 25, wherein a proximal end portion of the guidewire insertion aide is disposed outwardly from the blood outlet.

In an Example 27, the percutaneous circulatory support system of Example 23, wherein the guidewire insertion aide includes a through lumen.

In an Example 28, the percutaneous circulatory support system of Example 23, wherein the guidewire insertion aide includes a blind lumen.

In an Example 29, the percutaneous circulatory support system of Example 23, wherein the guidewire insertion aide includes a length in a range of 6.75 inches to 8 inches.

In an Example 30, a method of manufacturing a percutaneous circulatory support system includes providing a percutaneous circulatory support device, the percutaneous circulatory support device including a housing, an impeller carried in the housing, and a distal portion coupled to the housing; providing a guidewire insertion aide including a tapered distal end portion, the guidewire insertion aide being configured to receive a guidewire; and removably positioning the guidewire insertion aide in the housing and the distal portion of the percutaneous circulatory support device such that the tapered distal end portion is in contact with the distal portion of the percutaneous circulatory support device.

In an Example 31, the method of Example 30, wherein the guidewire insertion aide includes a through lumen.

In an Example 32, the method of Example 30, wherein the guidewire insertion aide includes a blind lumen.

In an Example 33, the method of Example 30, further including providing the guidewire detached from the guidewire insertion aide.

In an Example 34, the method of Example 30, wherein the distal portion includes a cannula, and removably positioning the guidewire insertion aide in the percutaneous circulatory support device includes contacting the tapered distal end portion against the cannula.

In an Example 35, the method of Example 34, wherein the percutaneous circulatory support device further includes a flexible distal extension coupled to the cannula opposite the housing, the flexible distal extension including a proximal end portion in contact with the cannula, and an opposite distal end portion, the tapered distal end portion of the guidewire loading aide being disposed proximally relative to the distal end portion of the flexible extension.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a side sectional view of an illustrative percutaneous circulatory support system including a percutaneous circulatory support device (also referred to herein, interchangeably, as a “blood pump”) and an illustrative guidewire insertion aide, in accordance with embodiments of the subject matter disclosed herein.

FIG. 2 is a detail side sectional view of the percutaneous circulatory support device and the guidewire insertion aide within line 2-2 of FIG. 1.

FIG. 3 is a side sectional view of the guidewire insertion aide of FIG. 1 and an illustrative guidewire, shown in phantom lines, in accordance with embodiments of the subject matter disclosed herein.

FIG. 4 is a side sectional view of another illustrative guidewire insertion aide and an illustrative guidewire, shown in phantom lines, in accordance with embodiments of the subject matter disclosed herein.

FIG. 5 is a diagram of a method of manufacturing a percutaneous circulatory support system, in accordance with embodiments of the subject matter disclosed herein.

FIG. 6 is a diagram of a method of using a percutaneous circulatory support system, in accordance with embodiments of the subject matter disclosed herein.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

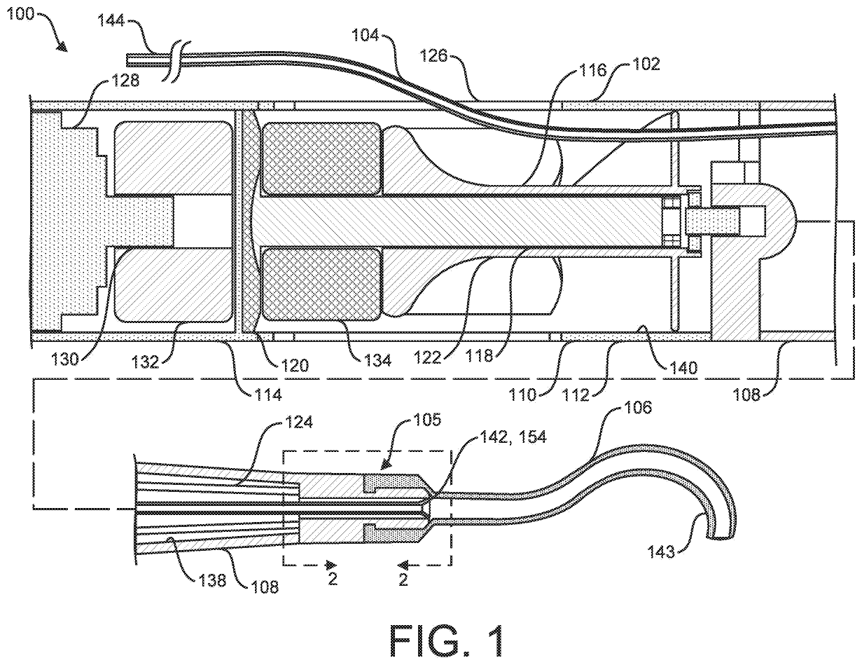

FIG. 1 depicts a partial side sectional view of an illustrative percutaneous circulatory support system 100 in accordance with embodiments of the subject matter disclosed herein. The system 100 includes a percutaneous circulatory support device 102 (also referred to herein, interchangeably, as a “blood pump”), a guidewire insertion aide 104, a guidewire (shown elsewhere), and an introducer sheath (not shown). The guidewire insertion aide 104 is initially partially disposed within the device 102 (that is, the guidewire insertion aide 104 is partially disposed within the device 102 before the system 100 is provided to a medical practitioner) and, as described in further detail herein, facilitates inserting the guidewire into the device 102 without potentially damaging delicate components of the device 102. After inserting the guidewire into the device 102, the guidewire and the introducer sheath may be used to percutaneously deliver the device 102 to a target location within a patient. More specifically, the device 102 may be translated along the guidewire to deliver the device 102 to the target location. The target location may be within the patient's heart. Alternatively, the device 102 may be delivered to a different target location within a patient.

With continued reference to FIG. 1, the device 102 generally includes a distal portion 105, more specifically a flexible distal extension 106 and a cannula 108, an impeller portion 110, and a proximal catheter (not shown). During operation, the device 102 may be positioned such that the distal extension 106 is located in close proximity of, or in contact with, the wall of the left ventricle of the patient, for example, in the location of the apex of the left ventricle. The cannula 108 may be partially positioned in the left ventricle and extend through the aortic valve of the patient. The impeller portion 110 and the catheter may be positioned in the aorta of the patient.

In some embodiments, the impeller portion 110 includes an impeller housing 112 and a motor housing 114. The impeller housing 112 and the motor housing 114 may be integrally or monolithically constructed. In other embodiments, the impeller housing 112 and the motor housing 114 may be separate components configured to be removably or permanently coupled. In other embodiments, the device 102 may lack the motor housing 114.

The impeller housing 112 carries an impeller assembly 116 therein. The impeller assembly 116 includes an impeller shaft 118 that is rotatably supported by at least one bearing, such as a bearing 120. The impeller assembly 116 also includes an impeller 122 that rotates relative to the impeller housing 112 to drive blood through the device 102. More specifically, the impeller 122 causes blood to flow from a blood inlet 124 of the cannula 108, through the cannula 108 and the impeller housing 112, and out of a blood outlet 126 formed on the impeller housing 112. In some embodiments and as illustrated, the impeller shaft 118 and the impeller 122 may be separate components, and in other embodiments the impeller shaft 118 and the impeller 122 may be integrated. In some embodiment and as illustrated, the inlet 124 and/or the outlet 126 may each include multiple apertures. In other embodiments, the inlet 124 and/or the outlet 126 may each include a single aperture. In some embodiments and as illustrated, the inlet 124 may be formed on a side portion of the cannula 108 and the outlet 126 may be formed on a side portion of the impeller housing 112.

With continued reference to FIG. 1, the motor housing 114 carries a motor 128, and the motor 128 is configured to rotatably drive the impeller 122 relative to the impeller housing 112. In the illustrated embodiment, the motor 128 rotates a drive shaft 130, which is coupled to a driving magnet 132. Rotation of the driving magnet 132 causes rotation of a driven magnet 134, which is connected to and rotates together with the impeller assembly 116. More specifically, in embodiments incorporating the impeller shaft 118, the impeller shaft 118 and the impeller 122 are configured to rotate with the driven magnet 134. In other embodiments, the motor 128 may couple to the impeller assembly 116 via other components. As noted herein, rotation of the impeller 122 causes blood to flow through the device 102.

In some embodiments, a controller may be operably coupled to the motor 128 and configured to control the motor 128. In some embodiments, the controller may be disposed within the motor housing 114. In other embodiments, the controller may be disposed outside of the motor housing 114 (for example, in a catheter handle, an independent housing, etc.). In some embodiments, the controller may include multiple components, one or more of which may be disposed within the motor housing 114. According to embodiments, the controller may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more Central Processing Units (CPUs), software, hardware, firmware, or any combination of these and/or other components. Although the controller is referred to herein in the singular, the controller may be implemented in multiple instances, distributed across multiple computing devices, instantiated within multiple virtual machines, and/or the like. In other embodiments, the motor 128 may be controlled in other manners.

With further reference to FIG. 1, the guidewire insertion aide 104 is initially partially disposed within the device 102. More specifically, the guidewire insertion aide 104 extends from within the distal portion 105, more specifically from a lumen 138 of the cannula 108, into an interior 140 of the impeller housing 112, and out of the blood outlet 126 of the impeller housing 112. Accordingly, a distal end portion 142 of the guidewire insertion aide 104 is initially disposed proximally relative to a distal end portion 143 of the distal extension 106, and an opposite proximal end portion 144 of the guidewire insertion aide 104 is initially disposed outwardly relative to the blood outlet 126.

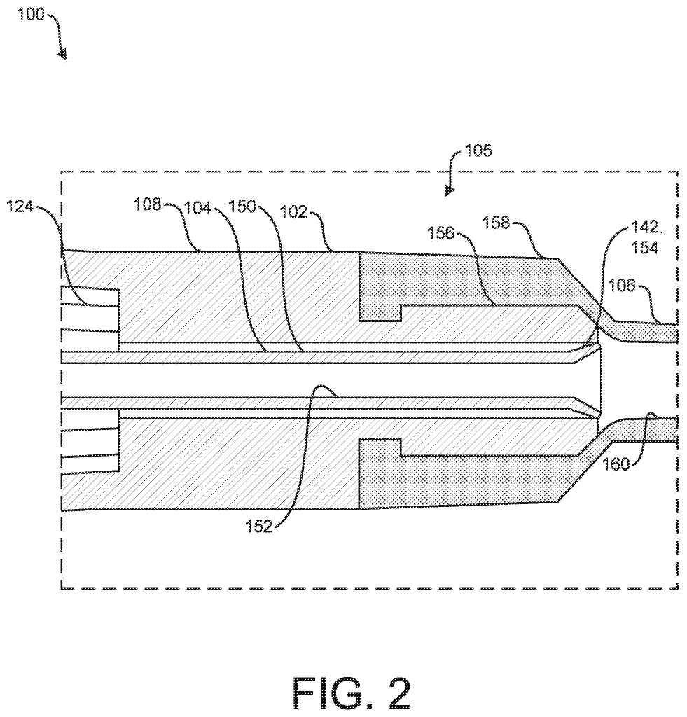

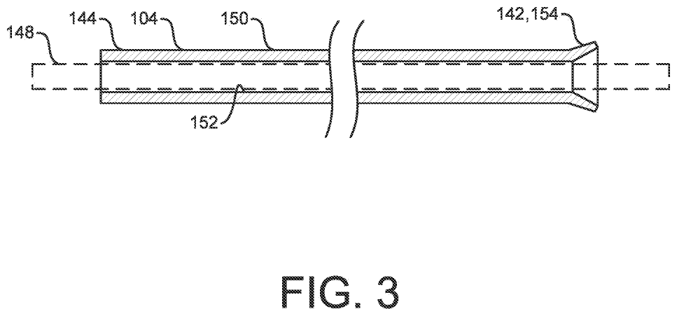

With reference now to FIGS. 2 and 3, the guidewire insertion aide 104 may be, generally, an elongated, flexible tube that is suitable for receiving a guidewire 148 (FIG. 3). The guidewire insertion aide 104 may be constructed of various appropriate materials, such as relatively soft and flexible biocompatible polymers (such as polyetheretherketone (PEEK)), metals, composites, or the like.

With continued reference to FIGS. 2 and 3, the guidewire insertion aide 104 generally includes a cylindrical wall 150 that defines a guidewire lumen 152 for receiving a guidewire 148 (FIG. 3). The guidewire insertion aide 104 also includes a coupling portion 154, which may be the distal end portion 142 of the guidewire insertion aide 104, that is enlarged radially outwardly relative to the wall 150 of the guidewire insertion aide 104. The coupling portion 154 initially contacts the distal portion 105 of the device 102 (FIG. 2), more specifically a flexible extension adapter 156 of the cannula 108 that mounts a proximal end portion 158 of the distal extension 106, to secure the guidewire insertion aide 104 within the device 102. In some embodiments and as illustrated, the coupling portion 154 includes a tapered or funnel shape. In other embodiments, the coupling portion 154 may take other forms.

In some embodiments and as illustrated, the lumen 152 of the guidewire insertion aide 104 is a through lumen (that is, a lumen including two openings, at the distal end portion 142 and the proximal end portion 144 of the guidewire insertion aide 104) through which a guidewire may extend. Interaction of a guidewire insertion aide 104 including such a through lumen 152 with a guidewire 148 is described in further detail herein. In other embodiments and as described in further detail herein, the lumen 152 may take other forms.

The guidewire insertion aide 104 may have any of various appropriate dimensions. For example, the guidewire insertion aide 104 may have a length in a range of 6.75 inches to 8 inches. As another example, the wall 150 of the guidewire insertion aide 104 may have a thickness in a range of 0.004 inches to 0.008 inches, more specifically 0.006 inches. As another example, the guidewire lumen 152 may have a diameter in a range of 0.015 inches to 0.025 inches, more specifically 0.020 inches. As another example, the enlarged coupling portion 154 may have a maximum outer diameter in a range of 0.041 inches to 0.042 inches. As yet another example, the opening of the guidewire lumen 152 at the enlarged coupling portion 154 may flare outwardly at an angle in a range of 52 degrees to 62 degrees. It also is contemplated that various types of guidewires may be used in the conjunction with the guidewire insertion aide 104. For example, different sizes of guidewires, including guidewires have diameters of 0.014 inches, 0.018 inches, or other commonly sized guidewires may be used with guidewire insertion aide 104. It is also contemplated that different sized guidewire insertion aides may be used to correspond to different sized guidewires.

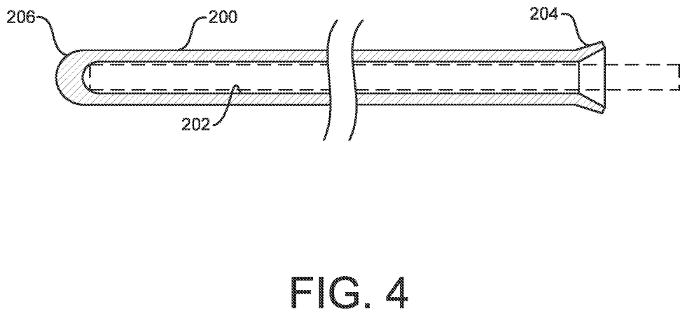

Guidewire insertion aides in accordance with embodiments of the subject matter disclosed herein may take other forms. For example, FIG. 4 depicts an illustrative guidewire insertion aide 200 in accordance with embodiments of the subject matter disclosed herein. The guidewire insertion aide 200 may form a part of the percutaneous circulatory support system 100 (shown elsewhere) by replacing the guidewire insertion aide 104 and being initially partially disposed in the device 102. The guidewire insertion aide 200 is generally similar to the guidewire insertion aide 104, except that the guidewire insertion aide 200 is a blind guidewire lumen 202 (that is, a lumen including an opening at the distal end portion 204 and a closed end at the proximal end portion 206). Accordingly, upon advancing the guidewire 148 proximally in the blind lumen 202 and contacting the closed proximal end portion 206, the guidewire 148 facilitates pushing the guidewire insertion aide 200 out of the device 102. Interaction of a guidewire insertion aide 200 including such a blind lumen 202 with a guidewire 148 is described in further detail herein.

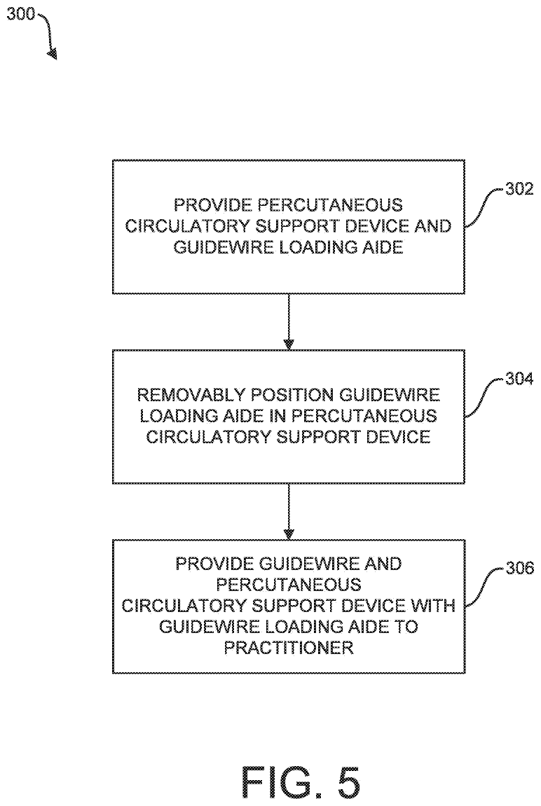

Referring to FIG. 5, a method 300 of assembling a percutaneous circulatory support system in accordance with embodiments of the subject matter disclosed herein may be generally as follows. The method 300 illustratively describes features of the system 100, the guidewire insertion aide 104, and the guidewire insertion aide 200, although it is understood that any system or guidewire insertion aide contemplated herein could be used in a similar manner. At block 302, the device 102 and the guidewire insertion aide 104 or 200 are provided. At block 304, the guidewire insertion aide 104 or 200 is removably positioned in the device 102 such that the guidewire insertion aide 104 or 200 extends from within the distal portion 105, more specifically from the cannula lumen 138, into an interior 140 of the impeller housing 112, and out of the blood outlet 126 of the impeller housing 112. As a result, the guidewire insertion aide 104 or 200 is disposed as shown in FIGS. 1 and 2, more specifically with the distal end portion 142 of the guidewire insertion aide 104 disposed proximally relative to a distal end portion 143 of the distal extension 106. As a result, the guidewire insertion aide 104 may not be in contact with the distal extension 106, which could alter the shape of the distal extension 106. The above actions may be conducted in a manufacturing environment (that is, not a medical environment). At block 306, the device 102 with the guidewire insertion aide 104 or 200 positioned in the device 102 is packaged for use by a medical practitioner (for example, a surgeon, a nurse, a medical environment administrator, or the like). The guidewire 148, detached from the guidewire insertion aide 104, may also be packaged for use by a medical practitioner.

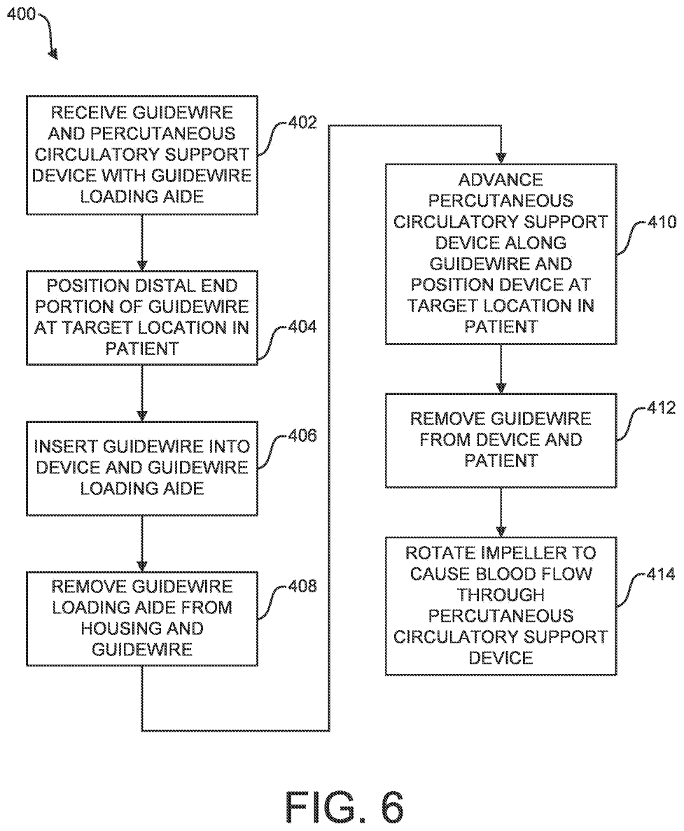

Referring to FIG. 6, a method 400 of using a percutaneous circulatory support system in accordance with embodiments of the subject matter disclosed herein may be generally as follows. The method 400 illustratively describes features of the system 100, the guidewire insertion aide 104, and the guidewire insertion aide 200, although it is understood that any system or guidewire insertion aide contemplated herein could be used in a similar manner. At block 402, the device 102 and the guidewire 148 are received by a medical practitioner. Upon receipt of the device 102, the guidewire insertion aide 104 or 200 is removably carried by the device 102 and disposed as shown in FIGS. 1 and 2. At block 404, the distal end portion of the guidewire 148 is inserted in the patient and advanced to the target location in the patient, such as the left ventricle. At block 406, the guidewire 148 is inserted into and advanced proximally through an extension lumen 160 of the distal extension 106 and into the lumen 152 or 202 of the guidewire insertion aide 104 or 200. More specifically and for the guidewire insertion aide 104, this action includes advancing the proximal end portion of the guidewire 148 through the through lumen 152 and outwardly from the proximal end portion 144 of the guidewire insertion aide 104, which is disposed outwardly from the blood outlet 126 of the device 102. More specifically and for the guidewire insertion aide 200, this action includes advancing the proximal end portion of the guidewire 148 in the blind lumen 202 and lightly contacting the closed proximal end portion 206 of the guidewire insertion aide 200. In both cases, the guidewire insertion aide 104 or 200 remains in the device 102 due to contact and resulting friction forces between the enlarged coupling portion 154 of the guidewire insertion aide 104 or 200 and the adapter 156 of the cannula 108. At block 408, the guidewire insertion aide 104 is removed from the device 102 and the guidewire 148 by overcoming the friction forces between the guidewire insertion aide 104 or 200 and the cannula 108. More specifically and for the guidewire insertion aide 104, the guidewire insertion aide 104 is pulled proximally along the guidewire 148 to move the guidewire insertion aide 104 out of the blood outlet 126 of the device 102 and detach the guidewire insertion aide 104 from the guidewire 148. More specifically and for the guidewire insertion aide 200, the guidewire 148 is advanced proximally through the device 102 to push the guidewire insertion aide 200 out of the blood outlet 126 of the device 102, and the guidewire insertion aide 200 is then pulled proximally along the proximal end of the guidewire 148 to detach the guidewire insertion aide 104 from the guidewire 148. At block 410, the device 102 is advanced along the guidewire 148 and positioned at the target location in the patient. At block 412, the guidewire 148 is pulled proximally and thereby removed from the device 102 and the patient. At block 414, the device is energized to rotate the impeller 122 relative to the impeller housing 112 and cause blood to flow through the device 102.

As described herein, guidewire insertion aides according to the present disclosure are initially disposed partially within percutaneous circulatory support devices and facilitate inserting guidewires into the devices. Because the guidewire insertion aides are positioned in the devices in a manufacturing environment (that is, not by a medical practitioner) and/or due to the materials of the guidewire insertion aides, embodiments according to the present disclosure avoid damage to delicate components of percutaneous circulatory support devices that may otherwise be caused by guidewire insertion. In addition, because the guidewire insertion aide does not extend into the distal extension of the device, the guidewire insertion aide will not alter the shape of the distal extension or otherwise affect the functionality of the distal extension.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A percutaneous circulatory support system, comprising:
a percutaneous circulatory support device comprising a housing, an impeller carried in the housing, and a distal portion coupled to the housing; and
a guidewire insertion aide removably carried in the housing and the distal portion of the percutaneous circulatory support device, the guidewire insertion aide comprising a tapered distal end portion initially in contact with the distal portion of the percutaneous circulatory support device, and the guidewire insertion aide configured to couple to a guidewire.

2. The percutaneous circulatory support system of claim 1, wherein the distal portion of the percutaneous circulatory support device comprises:
a cannula comprising a cannula lumen; and
a flexible distal extension comprising an extension lumen coupled to the cannula lumen;
wherein the tapered distal end portion of the guidewire insertion aide is initially disposed in the cannula lumen and in contact with the cannula.

3. The percutaneous circulatory support system of claim 2, wherein the cannula comprises a blood inlet and the housing comprises a blood outlet, and the guidewire insertion aide is configured to be removed from the housing through the blood outlet.

4. The percutaneous circulatory support system of claim 1, wherein the percutaneous circulatory support device comprises a blood inlet and a blood outlet, and the guidewire insertion aide is configured to be removed from the housing through the blood outlet.

5. The percutaneous circulatory support system of claim 4, wherein a proximal end portion of the guidewire insertion aide is disposed outwardly from the blood outlet.

6. The percutaneous circulatory support system of claim 1, wherein the guidewire insertion aide comprises a through lumen.

7. The percutaneous circulatory support system of claim 1, wherein the guidewire insertion aide comprises a blind lumen.

8. A percutaneous circulatory support system, comprising:
a percutaneous circulatory support device, comprising:
a housing;
an impeller carried in the housing;
a cannula coupled to the housing, the cannula comprising a distal adapter having a cannula lumen;
a distal extension coupled to the distal adapter; and
a guidewire insertion aide removably carried in the housing and the cannula lumen, the guidewire insertion aide comprising an enlarged coupling portion initially in contact with the distal adapter, and the guidewire insertion aide configured to couple to a guidewire.

9. The percutaneous circulatory support system of claim 8, wherein the enlarged coupling portion is a distal end portion of the guidewire insertion aide.

10. The percutaneous circulatory support system of claim 8, wherein the percutaneous circulatory support device comprises a blood inlet and a blood outlet, and the guidewire insertion aide is configured to be removed from the housing through the blood outlet.

11. The percutaneous circulatory support system of claim 10, wherein a proximal end portion of the guidewire insertion aide is disposed outwardly from the blood outlet.

12. The percutaneous circulatory support system of claim 8, wherein the guidewire insertion aide comprises a through lumen.

13. The percutaneous circulatory support system of claim 8, wherein the guidewire insertion aide comprises a blind lumen.

14. The percutaneous circulatory support system of claim 8, wherein the guidewire insertion aide comprises a length in a range of 6.75 inches to 8 inches.

15. A method of manufacturing a percutaneous circulatory support system, the method comprising:
providing a percutaneous circulatory support device, the percutaneous circulatory support device comprising a housing, an impeller carried in the housing, and a distal portion coupled to the housing;
providing a guidewire insertion aide comprising a tapered distal end portion, the guidewire insertion aide being configured to receive a guidewire; and
removably positioning the guidewire insertion aide in the housing and the distal portion of the percutaneous circulatory support device such that the tapered distal end portion is in contact with the distal portion of the percutaneous circulatory support device.

16. The method of claim 15, wherein the guidewire insertion aide comprises a through lumen.

17. The method of claim 15, wherein the guidewire insertion aide comprises a blind lumen.

18. The method of claim 15, wherein the method further comprises providing the guidewire detached from the guidewire insertion aide.

19. The method of claim 15, wherein the distal portion comprises a cannula, and removably positioning the guidewire insertion aide in the percutaneous circulatory support device comprises contacting the tapered distal end portion against the cannula.

20. The method of claim 16, wherein the percutaneous circulatory support device further comprises a flexible distal extension coupled to the cannula opposite the housing, the flexible distal extension comprising a proximal end portion in contact with the cannula, and an opposite distal end portion, the tapered distal end portion of the guidewire loading aide being disposed proximally relative to the distal end portion of the flexible extension.
